(19) United States
(12) Patent Application Publication
(10) Pub. No.: US 2021/0204840 A1
(43) Pub. Date: Jul. 8, 2021

(54) EAB BIOSENSORS FOR DETECTING SWEAT ANALYTES

(71) Applicant: Eccrine Systems, Inc., Cincinnati, OH (US)

(72) Inventors: Jacob A. Bertrand, Norwood, OH (US); Gavi Begtrup, Cincinnati, OH (US); Leila Safazadeh Haghighi, Cincinnati, OH (US); Jason Heikenfeld, Cincinnati, OH (US)

(73) Assignee: Eccrine Systems, Inc., Cincinnati, OH (US)

(21) Appl. No.: 16/087,342

(22) PCT Filed: Mar. 21, 2017
(86) PCT No.: PCT/US17/23399

§ 371 Date: Sep. 21, 2018

Related U.S. Application Data
(60) Provisional application No. 62/327,420, filed on Apr. 25, 2016.

(51) Int. Cl. A61B 5/1477; G01N 33/543; A61B 5/145; A61B 5/01; A61B 5/0533; A61B 5/00

(52) U.S. Cl. CPC A61B 5/1477; G01N 33/54366; A61B 5/14517; A61B 5/14539; A61B 5/14546; A61B 2562/0257; A61B 5/0533; A61B 5/4266; A61B 2562/0219; A61B 2562/0271; A61B 5/01

(57) ABSTRACT

Challenges of the sweat medium for electrochemical aptamer-based biosensor (“EAB sensor”) devices can be mitigated through aptamer selection, sensor/device configuration and physiological algorithms that account for the effects of (1) target analyte size, (2) potential concentration ranges, (3) sweat sample pH, and (4) sweat sample salinity. The disclosed invention includes a method of aptamer selection for use in an EAB sensor configured for use in a wearable sweat sensing device. The disclosed invention further provides a sweat sensing device configured to use EAB sensors to detect target analytes in a sweat sample.

### CROSS-REFERENCE TO RELATED APPLICATIONS

The present application claims priority to PCT/US17/23399, filed Mar. 21, 2017, and U.S. Provisional Application No. 62/327,420, filed Apr. 25, 2016; and has specification that builds upon PCT/US16/58356, filed Oct. 23, 2016, the disclosures of which are hereby incorporated herein by reference in their entirety.

### BACKGROUND OF THE INVENTION

Despite the many ergonomic advantages of sweat compared to other biofluids, particularly for sensing by wearable devices, sweat remains an underutilized source of biomarker analytes compared to blood, urine, and saliva. Upon closer comparison to other non-invasive biofluids, the advantages may even extend beyond ergonomics: sweat might provide superior analyte information. A number of challenges, however, have kept sweat from occupying its place among the preferred clinical biofluids. These challenges include very low sample volumes (nL to μL), unknown concentration due to evaporation, filtration and dilution of large analytes, mixing of old and new sweat, and the potential for contamination from the skin surface. More recently, rapid progress in wearable sweat sampling and sensing devices has resolved several of these challenges. However, this recent progress has also been limited to high concentration analytes (μM to mM) sampled at high sweat rates (>1 nL/min/gland) found in, e.g., athletic applications. Progress will be much more challenging as sweat biosensing moves towards detection of large, low concentration analytes (nM to pM and lower).

In particular, many known sensor technologies for detecting larger molecules are ill-suited for use in wearable sweat sensing, which requires sensors that permit continuous use on a wearer's skin. This means that sensor modalities that require complex microfluidic manipulation, the addition of reagents, the use of limited shelf-life components, such as antibodies, or sensors that are designed for a single use will not be sufficient for sweat sensing. What is needed is a stable, reliable bioelectric sensor that is sensitive to the target analyte in sweat, while being capable of multiple analyte capture events during the lifespan of the sensor. One solution to this problem is the use of electrochemical aptamer-based (“EAB”) sensor technology, such as is disclosed in U.S. Pat. Nos. 7,803,542 and 8,003,374. EAB sensors, however, have properties that make their use in wearable sweat sensing devices a considerable challenge. Therefore, the disclosed invention provides novel devices and methods to allow effective EAB sensor use in such devices.

### SUMMARY OF THE INVENTION

Challenges of the sweat medium for EAB sensor devices can be mitigated through aptamer selection, sensor/device configuration and physiological algorithms that account for the effects of (1) target analyte size, (2) potential concentration ranges, (3) sweat sample pH, and (4) sweat sample salinity. The disclosed invention includes a method of aptamer selection for use in an EAB sensor configured for use in a wearable sweat sensing device. The disclosed invention further provides a sweat sensing device configured to use EAB sensors to detect target analytes in a sweat sample.

### BRIEF DESCRIPTION OF THE DRAWINGS

The objects and advantages of the present invention will be further appreciated considering the following detailed descriptions and drawings in which:

FIGS. 1A and 1B represent an aptamer sensing element of the disclosed invention.

FIG. 2 depicts an embodiment of the disclosed invention.

FIG. 3 depicts an embodiment of the disclosed invention further including sweat sample treatment components for EAB sensing.

FIG. 4 is a view of an EAB response curve for varying sensor current output as a function of analyte concentration.

FIG. 5 is a view of an EAB response curve for varying sensor current output as a function of potential of hydrogen (pH).

### DEFINITIONS

“Continuous monitoring” means the capability of a device to provide at least one measurement of sweat determined by a continuous or multiple collection and sensing of that measurement or to provide a plurality of measurements of sweat over time.

“Sweat sampling rate” is the effective rate at which new sweat or sweat solutes, originating from the sweat gland or from skin or tissue, reaches a sensor which measures a property of sweat or its solutes. Sweat sampling rate, in some cases, can be far more complex than just sweat generation rate.

“Sweat stimulation” is the direct or indirect causing of sweat generation by any external stimulus, the external stimulus being applied to stimulate sweat. One example of sweat stimulation is the administration of a sweat stimulant such as pilocarpine. Going for a jog, which stimulates sweat, is only sweat stimulation if the subject jogging is jogging for the purpose of stimulating sweat.

“Sweat generation rate” is the rate at which sweat is generated by the sweat glands themselves. Sweat generation rate is typically measured by the flow rate from each gland in nL/min/gland. In some cases, the measurement is then multiplied by the number of sweat glands from which the sweat is being sampled.

“Chronological assurance” means the sampling rate or sampling interval that assures measurement(s) of analytes in sweat in terms of the rate at which measurements can be made of new sweat analytes emerging from the body. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5 to 30-minute lag time between analytes in blood emerging in interstitial fluid), but the resulting sampling interval (defined below) is independent of lag time, and furthermore, this lag time is inside the body, and therefore, for chronological assurance as defined above and interpreted herein, this lag time does not apply.

“Analyte-specific sensor” means a sensor specific to an analyte and performs specific chemical recognition of the analytes presence or concentration (e.g., ion-selective electrodes, enzymatic sensors, electro-chemical aptamer based sensors, etc.). For example, sensors that sense impedance or conductance of a fluid, such as sweat, are excluded from the definition of “analyte-specific sensor” because sensing impedance or conductance merges measurements of all ions in sweat (i.e., the sensor is not chemically selective; it provides an indirect measurement). Sensors could also be optical, mechanical, or use other physical/chemical methods which are specific to a single analyte. Further, multiple sensors can each be specific to one of multiple analytes.

“Sweat sensor data” means all the information collected by sweat system sensor(s) and communicated via the system to a user or a data aggregation location.

“Correlated aggregated sweat sensor data” means sweat sensor data that has been collected in a data aggregation location and correlated with outside information such as time, temperature, weather, location, user profile, other sweat sensor data, or any other relevant data.

“Measured” can imply an exact or precise quantitative measurement and can include broader meanings such as, for example, measuring a relative amount of change of something. Measured can also imply a binary measurement, such as ‘yes’ or ‘no’ type measurements.

“Sweat volume” is the fluidic volume in a space that can be defined multiple ways. Sweat volume may be the volume that exists between a sensor and the point of generation of sweat or a solute moving into or out of sweat from the body or from other sources. Sweat volume can include the volume that can be occupied by sweat between: the sampling site on the skin and a sensor on the skin where the sensor has no intervening layers, materials, or components between it and the skin; or the sampling site on the skin and a sensor on the skin where there are one or more layers, materials, or components between the sensor and the sampling site on the skin.

“Microfluidic components” are channels in polymer, textiles, paper, or other components known in the art of microfluidics for guiding movement of a fluid or at least partial containment of a fluid.

“Advective transport” is a transport mechanism of a substance or conserved property by a fluid due to the fluid's bulk motion.

As used herein, “diffusion” is the net movement of a substance from a region of high concentration to a region of low concentration. This is also referred to as the movement of a substance down a concentration gradient.

“Flow rate sensing component”, is any component or components which measure the flow rate of sweat in at least one portion of a sweat sensing or collecting device.

“Analyte” means a substance, molecule, ion, or other material that is measured by a sweat sensing device.

“Oligonucleotide” means a short nucleic acid polymer.

“Selectivity” means the ability of a sensor to respond to a target analyte.

“Sensitivity” means the change in output of the sensor per unit change in the parameter being measured. The change may be constant over the range of the sensor (linear), or it may vary (nonlinear).

“EAB sensor” means electrochemical aptamer-based biosensor that includes a plurality of aptamer sensing elements that produce a signal indicating target analyte capture, and which signal can be added to the signals of other such sensing elements, so that a signal threshold may be reached that indicates the presence of the target analyte.

“Aptamer” means a molecule that undergoes a conformation change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function.

### DETAILED DESCRIPTION OF THE INVENTION

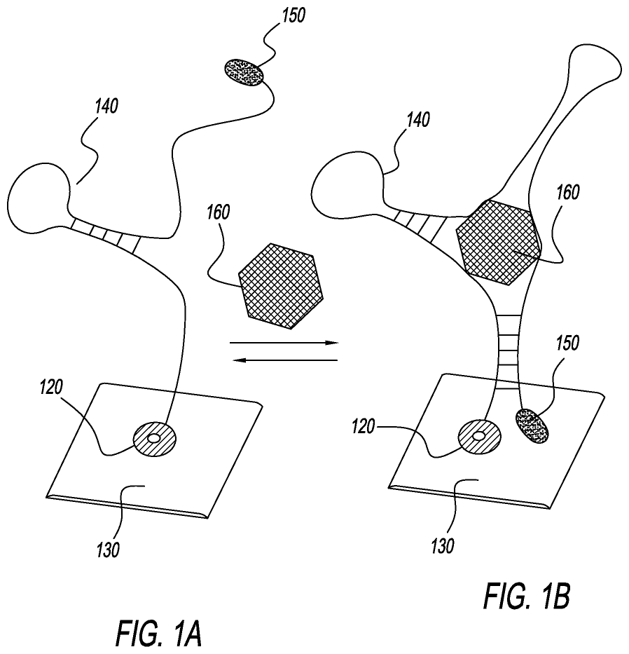

Electrochemical aptamer-based sensors for use in continuous sweat sensing are configured to provide stable sensor responses with a life cycle extensive enough for multiple analyte binding and release cycles. Such sensors include a plurality of individual aptamer sensing elements, as depicted in FIG. 1A, which can repeatedly detect the presence of a molecular target by capturing and releasing target analytes as they contact the aptamer. The sensing element includes an analyte capture complex 140 that has a first end covalently bonded to a sulfur molecule (thiol) 120, which is in turn covalently bonded to a gold electrode base 130. In other embodiments (not shown), the analyte capture complex may be bound to the electrode by means of an ethylenediaminetetraacetic acid (EDTA) strain, to improve adhesion in difficult sensing environments, such as sweat. The sensing element further includes a redox moiety 150 that may be covalently bonded to the aptamer 140 or bound to it by a linking section. In the absence of the target analyte, the aptamer 140 is in a first configuration, and the redox moiety 150 is in a corresponding first position relative to the electrode 130. When the device interrogates the sensing element using, e.g., square wave voltammetry (SWV), the sensing element produces a first electrical signal.

With reference to FIG. 1B, the aptamer 140 is selected to specifically interact with a target analyte 160, so that when the aptamer captures a target analyte molecule, the aptamer undergoes a conformation change that partially disrupts the first configuration, and forms a second configuration. The capture of the target analyte 160 accordingly moves the redox moiety 150 into a second position relative to the electrode 130. Now when the biofluid sensing device interrogates the sensing element, the sensing element produces a second electrical signal that is distinguishable from the first electrical signal. After a time interval of nanoseconds, milliseconds, seconds or longer, (the “recovery interval”), the aptamer releases the target analyte, and the aptamer will return to the first configuration, which will produce the corresponding first electrical signal when the sensing element is interrogated.

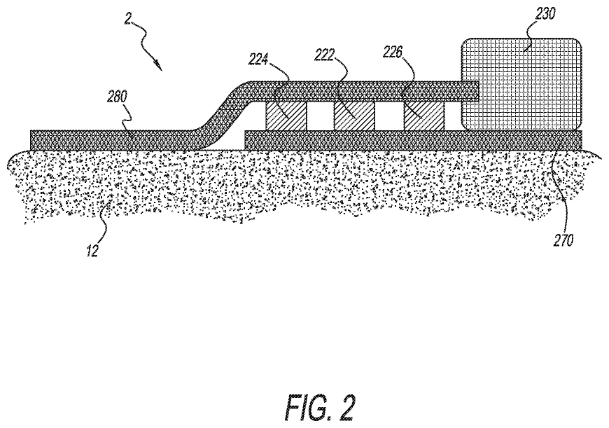

With reference to FIG. 2, a sweat sensing device 2 configured for use with an EAB sensor is depicted. The device includes a sweat-impermeable substrate 270 such as PET or Kapton, that carries a microfluidic wick or channel 280 which is in fluidic communication with skin 12, and functions to carry a sweat sample across at least one EAB sensor 222, 224, and secondary sensor(s) 226. The device is placed on skin 12, via an adhesive layer (not shown). Adhesives can be pressure sensitive, liquid, tacky hydrogels, which promote robust electrical, fluidic, and iontophoretic contact with skin. The wick may be made from, e.g., paper, Rayon, textiles, screen-printed hydrophilic nanocellulose, or other suitable materials. In some embodiments, the wick may instead be a microfluidic channel (not shown) made from a sweat impermeable material. The wick is in fluid communication with a pump 230, which facilitates wicking or osmotic flow of the sweat sample to, across, and away from the sensors. The pump 230 material is a hydrogel, textile, salt, desiccant, or other suitable material. The device includes at least one EAB sensor 222, 224, and one or more secondary sensors 226. Such secondary sensors 226 serve to improve EAB sensor interpretation, and include one or more of the following: an ion selective electrode sensor, a reference electrode, a temperature sensor, a skin impedance sensor, a capacitive skin proximity sensor, an accelerometer, a volumetric sweat rate sensor, a sweat conductivity sensor, and a galvanic skin response sensor.

While the disclosed embodiment may be sufficient for certain analytes, such as cortisol, that exist in sweat at higher concentrations, analytes existing in sweat at low concentrations, however, greatly complicate an EAB sensor's ability to provide reliable, continuous sensing. Concentration ranges for potential target analytes span from nM for cortisol, to μM for other hormones, to pM and even fM ranges for larger proteins. When target analyte concentrations are lower, EAB sensors will naturally have fewer capture opportunities, requiring greater sensitivity to ensure that the reduced capture opportunities are fully exploited.

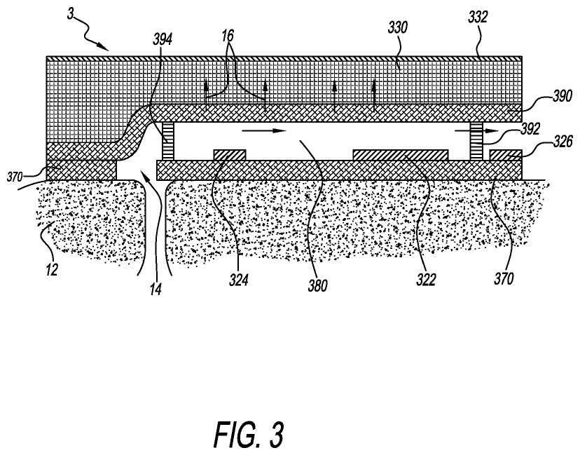

Therefore, with reference to FIG. 3, an embodiment is depicted that allows sample concentration for a sweat sensing device with an EAB sensor. The device 3 includes a sweat-impermeable substrate 370, that carries a concentration channel 380, at least one EAB sensor 322, a concentrating membrane 390, a pump 330, an optional secondary sensor 324, an optional post-sensor membrane 392, an optional pre-sensor membrane 394, and an optional protective covering 332. The device is placed on skin 12, via an adhesive layer (not shown). As the sweat sample 14 enters the device and flows into the concentration channel 380, water, and in some cases untargeted solutes, are drawn through the concentrating membrane 390, and into the pump 330. Such sample manipulation leaves the target analyte in the concentration channel 380, and effectively concentrates the sweat sample with respect to the target analyte.

The concentrating membrane is a dialysis membrane, or is an osmosis membrane permeable to ions and impermeable to small molecules and proteins, or may be a membrane that is at least permeable to water and impermeable to the target analyte. For example, a membrane with a 12 kDa molecular mass cutoff will retain solutes that are above 12 kDa, such as human serum albumin, which is 66.5 kDa. The material in the pump 330 may facilitate wicking or osmotic flow, and is a hydrogel, textile, salt, polyelectrolyte solution, or desiccant, such as MgSO₄. In some embodiments, the pump 330 would have a significantly greater volume than the concentration channel 380 to facilitate pH and salinity buffering of the sweat sample. Depending on the application, the target analyte may be concentrated at least 10×, 100×, or 1000× higher than its original concentration in sweat.

The optional post-sensor membrane 392, in some embodiments is made from similar material types as used for the concentrating membrane, is configured to pass fluid and solutes smaller than the target analyte, and causes the target analyte to further concentrate near the EAB sensor 322, where measurements are taken. In other embodiments, the post-sensor membrane 392 may simply substantially slow the flow of the sweat sample through the channel to aid in sample concentration. The optional pre-sensor membrane 394, also made from similar material types as used for the concentrating membrane, filters unwanted solutes, such as molecules larger than the target analyte, from the sweat sample before it reaches the EAB sensor 322.

Detection of the target analyte will be positively indicated when a sufficient number of EAB sensing elements captures a target analyte molecule, and produces a capture signal when interrogated by the device. The strength of the signal required to indicate the presence of the target in the sweat sample is known as the signal threshold. Signal threshold will vary by application, and will be set to achieve a desired predictive value that balances false positive indications and false negative indications. Some applications, such as screening the general population for a heart condition, may require very low false positive indications, and therefore would need to have a higher signal threshold, representing greater certainty of analyte presence. Other applications, such as preliminary screening for lead exposure in an at-risk population, may not require such high certainty, and could use a lower signal threshold. In other cases, for example, an EAB sensing element may have an aptamer that relatively weakly binds the target analyte, or the particular sweat sample may have challenging pH or salinity characteristics, or the target analyte may be very small. In each of these cases, the signal threshold would need to be relatively higher than in the converse case, all other factors being equal.

As with analytes found in sweat at low concentrations, small-molecule analytes tend to be relatively more difficult to detect with EAB sensors than larger analytes. Size ranges for potential target analytes range in size from about 300 Da for hormones to about 7 kDa for microRNA molecules to about 600 kDa for larger proteins to about 1000 kDa for the largest proteins. Other factors being equal, aptamers selected to capture smaller molecules will have inherently less specificity than those for larger molecules, since smaller molecules have relatively fewer unique binding site configurations. Also, aptamers will generally develop stronger bonds to larger molecules because of the greater number of binding sites available on such molecules. Consequently, small molecules form inherently less stable bonds with their complementary aptamers than do larger molecules. Further, sweat sample composition variabilities that tend to change bonding strength (such as pH and salinity) will generally have a greater effect on small molecule sensors than they will on larger molecule sensors. In addition, because smaller molecules have fewer binding sites, the difficulties of low concentration detection are even more pronounced for such sensors. Therefore, the signal threshold, or signal-to-noise ratio, for smaller molecules will be relatively higher to ensure an analyte capture event has occurred.

Compared to blood as a sensing medium, sweat presents certain advantages for aptamer sensing, chiefly because it is a more dilute, and hence a less electrically noisy, sensing environment. Sweat is also a less hostile environment for aptamers because there are fewer enzymes that can attack and degrade the aptamers. However, sweat also presents challenges that are not presented by the blood sensing environment. For example, whereas blood is a relatively homogenous solution, sweat is more variable in solute concentration and distribution. This variability correlates to greater ranges in pH and salinity values than an aptamer sensor would experience in blood. Sweat's variability in pH and salinity presents a significant challenge for the use of EAB sensors for continuous analyte monitoring.

The challenges of the sweat medium for EAB biosensing can be mitigated through aptamer selection, sensor/device configuration and physiological algorithms that account for the effects of (1) target analyte size, (2) potential concentration ranges, (3) sweat sample pH, and (4) sweat sample salinity. These factors are important determinants of core EAB sensor characteristics, such as sensitivity, selectivity, linear range and recovery time.

As a first order consideration, target analyte size can greatly affect EAB sensor performance, especially in combination with pH and salinity variability. As discussed above, aptamers will generally develop stronger bonds to larger molecules because of the greater number of bonding sites available on such molecules. This effect may not apply to molecules that are substantially larger than the aptamer, because a larger molecule may require the aptamer to undergo significant conformational change to effect binding, thereby lowering the free energy gain and reducing the spontaneity of the binding. Therefore, the aptamer selection process will need to consider the inherent bonding strength of aptamers for larger versus smaller target molecules.

Similarly, analyte size will also affect EAB sensor recovery time, which will impact the device's chronological assurance. For reference, see PCT/US2014/061098, incorporated by reference herein in its entirety. For example, aptamer sensing elements configured to detect small molecules, due to the relatively small number of binding sites, will tend to have shorter recovery times (seconds or faster), which effect is enhanced as the analyte concentration decreases. Therefore, small molecule EAB sensors will need little or no recovery time or sensor regeneration in between sensing events. By contrast, for large molecule EAB sensors, the increased binding strength between the aptamer and the target molecule will tend to result in longer sensor recovery times, which could reach into the hour range or longer. Hydrophilicity/hydrophobicity will also influence aptamer sensing element recovery times. For example, a molecule that has a net hydrophobic character will be less likely to release from the aptamer if it is bound in a hydrophobic pocket of the aptamer. Therefore, to arrive at a chronologically assured sweat sampling rate, not only will the device account for sweat rate and sweat volume, but must also consider the recovery time needed by the EAB sensors. Fortunately, most protein (large molecule) concentrations change very slowly in the body, and sweat sampling intervals in the multiple hour range will be acceptable for most applications.

For those applications that require shorter sampling intervals, some form of sensor regeneration may be required. For example, EAB regeneration may be accomplished by placing a heating component in proximity to the sensor, which would cause the target analyte to detach from the aptamer and return to solution. Another regeneration means would comprise the introduction of fluid to the EAB sensor, for example by means of a small-volume fluid reservoir containing neutral pH or sweat similar fluid. The fluid reservoir may be located upstream of the EAB sensor, and when required, the sweat sensor device could open a microfluidic valve to release the fluid, which would be pulled across the aptamer sensing elements by wicking pressure, thereby causing the analytes to release from the aptamers and return to solution.

Secondly, the sweat sample concentration ranges of the target analyte will affect EAB sensing applications. When target analyte concentrations are lower, EABs will naturally have fewer capture opportunities, requiring greater sensitivity to ensure that the reduced capture opportunities are fully exploited. Therefore, aptamer selection will have to tend toward greater sensitivity for low concentration analytes. In addition, because smaller molecules have fewer binding sites, the effect of low concentration is more pronounced for these analytes, and aptamer sensors may not have the affinity to function for small, low concentration analytes.

While analyte size and concentration will greatly influence EAB performance, the sweat sample environment variability in pH and salinity must be considered in their own right, and must also inform the effects of analyte size and concentration. Due to the relative magnitude of pH variability—salinity ranges from 10 to 100 mM (≈4×), while pH ranges from 4.5 to 7.0 (≈300×)—pH is usually the more influential factor. pH primarily affects EAB sensor performance through the reactive potential of the redox moiety that produces the electrical signal indicating analyte capture. pH also influences the bonding characteristics of the aptamer and analyte by dictating the degree of protonation of the hydrogen bonding sites within both the analyte and aptamer. With a lower pH sweat sample (more H+ in solution), there will be fewer possible hydrogen bonds that can be established between the aptamer and analyte, because more H+ ions from solution will fill the open bonding sites. Lower pH also decreases the mobility of ions in solution, and hence makes it more difficult for larger molecules in a sweat sample to move toward the EAB sensor. Therefore, variability in sweat pH should be mitigated, for example, through using a redox moiety that has a stable redox potential over the expected pH range, aptamer selection, or by managing the pH of the sweat sample near the EAB sensor.

Salinity affects EAB sensor performance through random small molecule binding. When an EAB is in the presence of a fluid sample, a certain number of bonding sites on the aptamer will bond with random ions (e.g., H+, Na+, Cl−, K+) in solution. These random binding events affect EAB performance by altering both the analyte capturing characteristics of the aptamer, as well as the structures of the unfolded and folded aptamer. Random binding tends to inhibit analyte capture by occupying some of the binding sites that should bind with the target analyte, giving the aptamer fewer bonds with the target analyte and resulting in a weaker bond when the analyte is captured.

Random binding also changes the shape of the EAB structure. When the aptamer is unbound to a target analyte, the redox molecule is in a first position relative to the gold electrode, and when a target analyte is captured, the redox molecule moves to a second position relative to the electrode. Random binding events change the first position of the redox couple, thereby altering the starting signal produced by the aptamer sensing element. In addition, randomly bound small molecules also change the aptamer's folding characteristics when a target analyte is captured, causing the aptamer to fold to a different second position than it would without the randomly bound ions. As a result, the signal difference produced by the aptamer sensing element when in its bound and unbound states may be relatively smaller, or may even reverse in valence. In addition, highly saline sweat samples may cause the aptamer sensing element to experience salting out, a condition in which larger proteins fall out of solution due to the salinity. The EAB's capture signal must therefore be corrected for aptamer fouling caused by randomly bound small molecules and salting out.

For example, a sweat sensing device configured with EAB sensors for a target analyte may derive an analyte concentration from the number of aptamer sensing elements that register an analyte capture event, which is inferred from the strength of the electrical signal generated by the sensor array. However, in the presence of low pH sweat, random binding events would alter the folding structure of the aptamer sensing elements, diminishing the array's electrical response. If left uncorrected, this reduced electrical response would be interpreted as a lower analyte concentration. Because of this inability to distinguish electrically between the number of responsive aptamer sensing elements and the degree of folding experienced by those aptamer sensing elements, corrections for pH and salinity may be necessary to correctly interpret analyte concentrations using EAB sensors. The described folding structure changes caused by random binding events are difficult to determine predictively, so aptamer behavior in different pH and salinity environments will usually be experimentally derived.

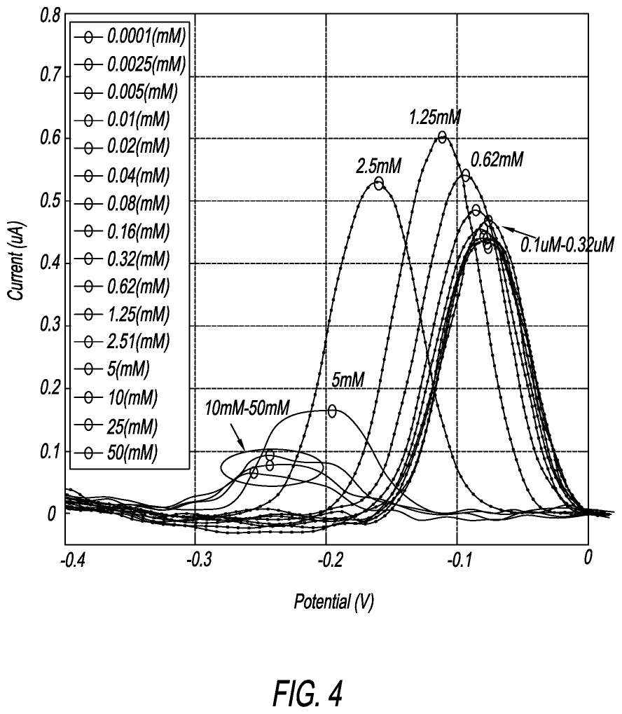

While an individual aptamer's reaction to changes in pH, salinity or other factors are difficult to predict, the type and scale of such effects can be illustrated. With reference to FIG. 4, the effect of pH variation on the output of an EAB kanamycin sensor is graphically depicted. A sweat-like solution was doped with increasing concentrations of kanamycin, which decreases the pH of the solution. The EAB sensor configured to detect kanamycin shows a consistent current response of about 0.45 μA up to about 0.32 mM of kanamycin in solution. However, increasing kanamycin concentrations initially raise the current output to a peak of 0.6 μA at 1.25 mM, and then the output decreases to less than 0.1 μA for concentrations above 10 mM. Absent a correction for pH, a sweat sensing device could incorrectly interpret these increased or decreased output currents as indicative of analyte concentration.

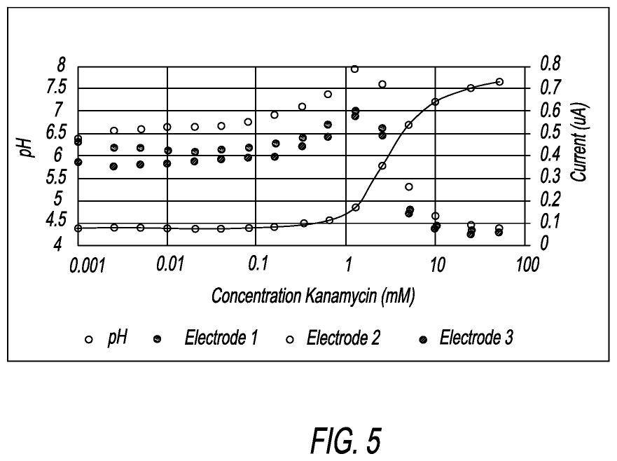

With reference to FIG. 5, this change in kanamycin EAB sensor output is further depicted in terms of pH. As in the previous figure, sensor output remains fairly constant as long as pH remains constant at 4.5. However, starting at about 0.5 mM of kanamycin, pH and sensor output begin to increase until peaking at about 4.8 pH. Then, as pH continues to increase toward 7, EAB sensor output drops sharply until output current is below 0.1 μA. As shown by the above example, pH can significantly affect the useful range of an EAB sensor. The kanamycin sensor demonstrates rapidly degrading responses to concentrations above 0.62 mM. Increases in salinity may similarly effect EAB sensor useful range. However, salinity will primarily act upon the aptamer itself, rather than the redox moiety, and because of the smaller range of variation for salinity in sweat, is expected to have a smaller effect on sensor response.

As shown, pH can have a dramatic effect on sensor output current. Most of the observed effects of pH on the kanamycin sensor are attributable to the methylene blue redox moiety, which experiences a proton transfer as part of its redox reaction. The electrical potential of this reaction is highly dependent on pH. Since sweat pH can naturally vary from between 4.5 and 7.0, its effects on the EAB sensor should be accounted for through redox moiety selection, aptamer selection, and or physiological algorithm design. The selection of a pH-independent redox moiety is disclosed in U.S. Provisional Application No. 62/403,341, filed Oct. 3, 2016, which is hereby incorporated herein in its entirety.

In addition to redox moiety selection, EAB sensor development should account for the potential effects of pH and salinity through aptamer selection. Through various methods known in the art of aptamer selection, chiefly different versions of Systematic Evolution of Ligands by Exponential Enrichment (“SELEX”) techniques, an aptamer for binding a target analyte is selected to reliably detect meaningfully small changes in concentration values, and have a linear response to concentration changes over the range expected for the target analyte in a sweat sample. At times, a particular aptamer may not be able to provide linear response over the entire expected analyte concentration range, and therefore a plurality of aptamers may be chosen to cover the concentration range. The aptamer is also chosen for its affinity to the target molecule, such that the molecule will be reliably captured, giving the EAB the desired selectivity and specificity, but the target will not bind so tightly that the EAB has slow regeneration characteristics, which tend to lower chronologically assured sampling rates. In some cases, aptamers selected for particular analytes or applications may have significant cross-selectivity with other molecules, for example an aptamer selected to capture luteinizing hormone (“LH”) may also capture follicle stimulating hormone (“FSH”). To improve performance, a plurality of aptamers may be selected for LH each of which have different cross-selectivity with FSH, allowing the device to correct for the contribution of FSH to the LH measurement.

One means of conducting aptamer selection to account for sweat sample pH and salinity variability is to conduct the SELEX, or other suitable process, in a sweat-like matrix. Alternately, a criterion for aptamer selection could be optimal EAB sensor performance in sweat for an expected range of pH and salinity values, or aptamer selection may account for EAB sensor performance in challenging or worst-case pH and salinity conditions. The aptamer's performance characteristics for the particular analyte and projected concentration range should also be considered. In addition, the likely use case for the sensor, such as high sweat rates for athletic applications, which will tend to have lower salinity/higher pH, should also be characterized, since an aptamer selected for optimal performance in low pH may have altered performance in higher pH sweat.

In addition to judicious aptamer selection, EAB sensors may be configured to provide improved performance in the particular sweat sensing environment characterizing the application and or target analyte. For example, some target analytes may have a broad concentration range, therefore a plurality of aptamer sensing elements may be used, each selected for optimal performance in a portion of the concentration range. To optimize detection of low concentration analytes, EAB sensors may be electromagnetically shielded to reduce the effects of electrical noise, thereby enabling the EAB sensor to improve sensitivity.

With further reference to FIG. 3, the depicted embodiment includes several features intended to mitigate pH and salinity variabilities in the sweat medium. Some embodiments of the disclosed invention may use the concentrating membrane 390 and pump 330 to maintain the sweat sample at a pH or salinity level while in contact with the EAB sensor(s) 322. Using an appropriate material in the pump 330, such as a polyelectrolyte solution with a higher pH than the sweat sample, can adjust the sweat sample pH within the channel 380. Other solutions, such as a hydrogel having very low NaCl content, can reduce the sweat sample salinity as needed. Some embodiments may feature one draw solution for pH buffering and a separate draw solution for salinity buffering. In other embodiments, the channel 380 itself contains a neutral pH or saline fluid surrounding the EAB sensor 322 to achieve conditions for optimal sensing.

EAB sensor(s) 322, may be protected from enzymes by using the pre-filter 394 as a microfluidic filtering system that allows the free passage of molecules in the size range of the target analyte, and blocks molecules in the enzyme size range. Alternately, EAB sensor robustness can be improved by selecting left-hand oriented aptamers, which will suffer less degradation due to enzymes, such as RNases and DNases. The pre-filter 394 can also protect the EAB sensor from ethanol and other alcohols by removing them from the sample.

Various other effects upon the sweat sample also may be accomplished by using complementary materials for the concentrating membrane, the post-sensor membrane and the pre-sensor membrane. For example, one membrane could be an anion exchange membrane, e.g., a modified poly(phthalazinon ether sulfone ketone), and another could be a cation exchange membrane, e.g., nafion or poly(vinyl alcohol)-SiO₂, or the membranes could all be dialysis membranes with different mass cutoffs. Some embodiments may filter out contents of the sweat sample to allow short lifespan analytes to survive in sweat and to reach the EAB sensor, for example, vasopressin detection may benefit from filtering configurations designed to preserve the analyte in sweat. Such filtering configurations could remove competing molecules from the sweat sample, e.g., estradiol may be removed to improve estrogen detection. Other filtering techniques, such as electrical charge-based filtering, hydrophobicity, hydrophilicity, lipophobicity, lipophilicity, etc., may also be used to protect either the aptamer or the target analyte to improve EAB sensor performance for a particular analyte or application environment. Some embodiments may include one or more pre-filters for each EAB sensor, so that the sweat sample may be tailored for each EAB sensor. Other embodiments include a plurality of pre-filters to allow the sweat sample to be filtered in stages.

EAB sensors can be calibrated for operation in sweat samples with varying salinities and pH values. As a first step in such a calibration process, EAB sensor performance is characterized for an expected range of sweat sample salinity and pH values. For example, the performance characterization would, at a minimum, include an assessment of the EAB sensor's current output when exposed to an expected range of target analyte concentrations. The EAB sensor's current output for the various concentration levels would then be duplicated throughout the expected salinity range (10 to 100 mM) and pH range (4.5 to 7.0). The relationship between salinity and pH during the performance characterization may reflect the expected correlations as found in the body, and may also include significant performance benchmarks for the aptamer being used, such as an optimal (or worst-case) pH and salinity. Once an EAB sensor's performance is adequately characterized for the expected pH and salinity ranges, the sensor's output may be corrected algorithmically by measuring the sweat sample salinity and pH, and applying an appropriate correction factor. Sweat sample salinity can be easily measured with ion-selective electrode sensors placed in proximity to the EAB sensor(s), and similarly, pH may be measured by pH sensors placed in proximity to each EAB sensor.

This has been a description of the disclosed invention along with a preferred method of practicing the disclosed invention, however the invention itself should only be defined by the appended claims.

What is claimed is:

1. A method, comprising:
selecting an aptamer for use in an electrochemical aptamer-based biosensor (“EAB sensor”), wherein said sensor is configured to measure a characteristic of a target analyte in sweat, said selection comprising:
defining at least one performance parameter for the EAB sensor;
using a size of the target analyte to define the at least one performance parameter;
using a sweat concentration range of the target analyte to define the at least one performance parameter;
using a sweat sample potential of hydrogen (pH) to define the at least one performance parameter;
using a sweat sample salinity to define the at least one performance parameter; and
using the at least one performance parameter to select an aptamer, wherein the aptamer is configured to interact with the target analyte.

2. The method of claim 1, wherein the at least one performance parameter is one of the following: a specificity to the target analyte, a sensitivity to the target analyte, a cross-selectivity to molecules that are not the target analyte, a linear range of detection, an aptamer recovery time.

3. The method of claim 1, wherein the at least one performance parameter is optimized for one of the following factors: a target analyte sized, sweat concentration range of the target analyte, a sweat sample pH, and a sweat sample salinity.

4. (canceled)

5. The method of claim 1, wherein the size of the target analyte is one of the following: 100 to 1000 Daltons (Da), 1 to 10 kDa, 10 to 1000 kDa, greater than 1000 kDa.

6. (canceled)

7. The method of claim 1, wherein the aptamer is one of the following: a DNA aptamer, an RNA aptamer, an XNA oligonucleotide, a left-hand aptamer, a spiegelmer, a peptide aptamer, an affimer, and a modified aptamer.

8. A sweat-sensing device configured to be placed on a skin surface of a wearer of the sweat-sensing device, the sweat-sensing device comprising:
at least one analyte-specific electrochemical aptamer-based biosensor configured to take one or more measurements of at least one target analyte in a sweat sample;
at least one secondary sensor;
a microfluidic channel in fluid communication with the skin surface of the wearer of the sweat-sensing device, the at least one analyte-specific electrochemical aptamer-based biosensor, and with the at least one secondary sensor, the microfluidic channel including an upstream portion and a downstream portion; and
a microfluidic pump in fluid communication with the microfluidic channel, the microfluidic pump configured to cause the sweat sample to flow through the microfluidic channel from the upstream portion, across the at least one analyte-specific electrochemical aptamer-based biosensor, and to the downstream portion.

9. The sweat-sensing device of claim 8, wherein the at least one secondary sensor is one of: an ion selective electrode sensor, a reference electrode, a temperature sensor, a skin impedance sensor, a capacitive skin proximity sensor, an accelerometer, a volumetric sweat rate sensor, a sweat conductivity sensor, and/or a galvanic skin response sensor.

10. The sweat-sensing device of claim 8, wherein the at least one secondary sensor measures a pH of the sweat sample.

11. The sweat-sensing device of claim 8, wherein the at least one secondary sensor measures sweat sample salinity.

12. The sweat-sensing device of claim 8, further including at least one sweat sample concentrator configured to concentrate the sweat sample with respect to at least one target analyte, wherein the at least one sweat sample concentrator receives an unconcentrated sweat sample that contains the at least one target analyte at a first molarity, and wherein the at least one sweat sample concentrator increases a concentration of the at least one target analyte within the sweat sample to second molarity that is at least 2 times higher than the first molarity.

13. The sweat-sensing device of claim 12, wherein the at least one sweat sample concentrator includes a selectively-permeable membrane that is permeable to water and impermeable to the at least one target analyte.

14. (canceled)

15. (canceled)

16. The sweat-sensing device of claim 13, wherein the selectively-permeable membrane protects the at least one analyte-specific electrochemical aptamer-based biosensor or the at least one target analyte from an enzyme.

17. (canceled)

18. The sweat-sensing device of claim 13, wherein the selectively-permeable membrane is configured to remove molecules with cross-selectivity to the at least one target analyte from the sweat sample.

19. The sweat-sensing device of claim 13, wherein the selectively-permeable membrane is hydrophobic, hydrophilic, lipophobic, or lipophilic.

20. (canceled)

21. The sweat-sensing device of claim 8, further comprising:
an electromagnetic shielding component configured to reduce electrical noise included in a measurement of the at least one analyte-specific electrochemical aptamer-based biosensor.

22. The sweat-sensing device of claim 8, further comprising:
a regenerating component configured to supply heat to the at least one analyte-specific electrochemical aptamer-based biosensor to regenerate the at least one analyte-specific electrochemical aptamer-based biosensor.

23. (canceled)

24. The sweat-sensing device of claim 22, wherein the regenerating component comprises:
a fluid reservoir containing a fluid, wherein the fluid reservoir is positioned in fluid communication to the upstream portion of the microfluidic channel, and wherein a pH of the fluid is neutral; and
a microfluidic valve configured to enable selectable release of the fluid, wherein when the microfluidic valve is opened, the fluid flows from the upstream portion of the microfluidic channel toward the downstream portion of the microfluidic channel and across the at least one analyte-specific electrochemical aptamer-based biosensor.

25. A method of using the sweat-sensing device of claim 8 to detect the at least one target analyte in a sweat sample, the method comprising:
defining a signal threshold indicating a threshold signal strength to detect a presence of the at least one target analyte, the signal threshold being defined based on at least one of: a predictive value for a device application, a size of the at least one target analyte, a sweat concentration range of the at least one target analyte in the sweat sample, potential of hydrogen (pH) of the sweat sample, and a salinity of the sweat sample,
detecting a signal from the at least one analyte-specific electrochemical aptamer-based biosensor or the at least one secondary sensor, the signal indicating that the at least one target analyte has been detected in the sweat sample;
comparing the signal to the signal threshold;
determining, based on comparing the signal to the signal threshold, that the signal threshold is reached; and
reporting that the at least one target analyte has been detected in response to determining that the signal threshold is reached.

26. The method of claim 25, further comprising:
determining a sweat sampling rate, where the sweat sampling rate is based at least on an electrochemical aptamer-based biosensor recovery time.

27. The method of claim 25, further comprising:
determining a concentration of the at least one target analyte in the sweat sample based on at least one of: the size of the at least one target analyte, the sweat concentration range of the at least one target analyte in the sweat sample, the pH of the sweat sample, and the salinity of the sweat sample.
